(19) United States
(12) Patent Application Publication
(10) Pub. No.: US 2004/0260283 A1
(43) Pub. Date: Dec. 23, 2004

(54) Multi-axis spinal fixation device

(76) Inventors: Shing-Cheng Wu, Taipei (TW); Po-Quang Chen, Taipei (TW)

Correspondence Address: BROWDY AND NEIMARK, P.L.L.C., 624 NINTH STREET, NW, SUITE 300, WASHINGTON, DC, 20001-5303, US

(21) Appl. No.: 10/464,751

(22) Filed: Jun. 19, 2003

(51) Int. Cl. A61B 17/56

(52) U.S. Cl. 606/61

(57) ABSTRACT

A multi-axis spinal fixation device includes a supporting pole having a threaded shaft at one end and a connecting seat at the other, wherein the connecting seat comprises a U-shaped recess; a brace rod installed across the U-shaped recess; a fastener fastening the brace rod in the recess; and a stabilizing piece disposed between the supporting pole and the fastener, the stabilizing piece having a curved surface at its one end facing the brace rod such that the stabilizing piece is in surface contact with the brace rod.

### BACKGROUND OF THE INVENTION

1. Field of the Invention

The present invention relates to a multi-axis spinal fixation device. More particularly, the present invention relates to a spinal fixation device having an elongate brace member being fastened to a supporting pole of the spinal fixation device with a curved seat fastener, which is in surface contact with the elongate brace member.

2. Description of the Prior Art

FIG. 1 illustrates a cross section of a conventional spinal fixation device. The conventional spinal fixation device comprises a supporting pole 1A. The supporting pole 1A typically has a threaded portion 11A as a bone screw for implanting into the spine. The conventional spinal fixation device further includes a threaded fastener 2A, which is used to fasten an elongate brace member 13A, which is used for fixation and/or manipulation of the spine. As illustrated, the elongate brace member 13A is disposed in a recess 12A defined at the cylindrical upper end of the supporting pole 1A. The threaded fastener 2A comprises an outer nut 21A and an inner threaded piece (screw) 22A. With the outer nut 21A and the inner threaded piece 22A, which are secured to the cylindrical upper end of the supporting pole 1A, the elongate brace member 13A therein is fastened, thereby connecting two such spinal fixation devices. However, the above-described prior art spinal fixation device has a drawback in that the threaded fastener 2A cannot tightly lock the brace member 13A in place since the plane bottom of the inner threaded piece 22A of the threaded fastener 2A is in line contact with the underlying brace member 13A. In other words, the conventional set screws can work their way loose when confronted with continuous micro-motion of the spine. This sometimes causes the failure of the spinal fixation device.

Thus, there is a strong need for an improved spinal fixation device which is reliable and is capable of implementing a multi-axis movement.

### SUMMARY OF THE INVENTION

Accordingly, the main objective of the invention is to provide an improved multi-axis spinal fixation device to solve the above-mentioned problems. According to the present invention, the multi-axis spinal fixation device has an elongate brace member capable of being securely fastened to a supporting pole of the spinal fixation device with a curved seat fastener, which is in surface contact with the surface of the elongate brace member.

In accordance with the present invention, a multi-axis spinal fixation device is provided. The multi-axis spinal fixation device comprises a supporting pole having a threaded shaft at one end and a connecting seat at the other, wherein the connecting seat comprises a U-shaped recess; a brace rod installed across the U-shaped recess; a fastener fastening the brace rod in the recess; and a stabilizing piece disposed between the supporting pole and the fastener, the stabilizing piece having a curved surface at its one end facing the brace rod such that the stabilizing piece is in surface contact with the brace rod.

From another aspect of the present invention, a multi-axis spinal fixation device comprises: a supporting pole having a threaded shaft at one end and a connecting seat at the other; a hollow sleeve, having a recess at its upper end, used to movably sleeve the connecting seat and hold the connecting seat therein; a brace rod laterally installed across the recess; and a fastener screwed in the sleeve, thereby immobilizing the brace rod within the recess of the sleeve, wherein the fastener is in surface contact with the brace rod.

The fastener further comprises an outer nut, an inner nut, an upper stabilizing piece, and a lower stabilizing piece, the upper stabilizing piece is located between the brace rod and the inner nut, and the lower stabilizing piece is located between the brace rod and the connecting seat. The brace rod is clamped by the upper stabilizing piece and lower stabilizing piece, the upper and lower stabilizing pieces have curved surface, which are engaged with the brace rod such that the upper and lower stabilizing pieces are in surface contact with the brace rod.

Other objects, advantages and novel features of the invention will become more clearly and readily apparent from the following detailed description when taken in conjunction with the accompanying drawings.

### BRIEF DESCRIPTION OF THE DRAWINGS

FIG. 1 illustrates a cross section of a conventional spinal fixation device.

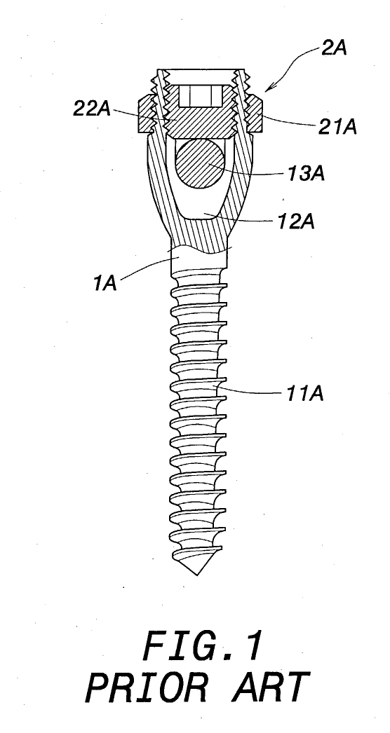

FIG. 2 is an exploded diagram illustrating a spinal fixation device according to one preferred embodiment of this invention.

FIG. 3 is a perspective view of the spinal fixation assembly of FIG. 2.

FIG. 4 is a cross-sectional diagram illustrating the spinal fixation device according to the present invention.

FIG. 5 is a plane view of this invention, when in use.

FIG. 6 is an exploded diagram showing a second preferred embodiment according to the present invention.

FIG. 7 is a cross-sectional view of FIG. 6.

### DETAILED DESCRIPTION OF THE PREFERRED EMBODIMENTS

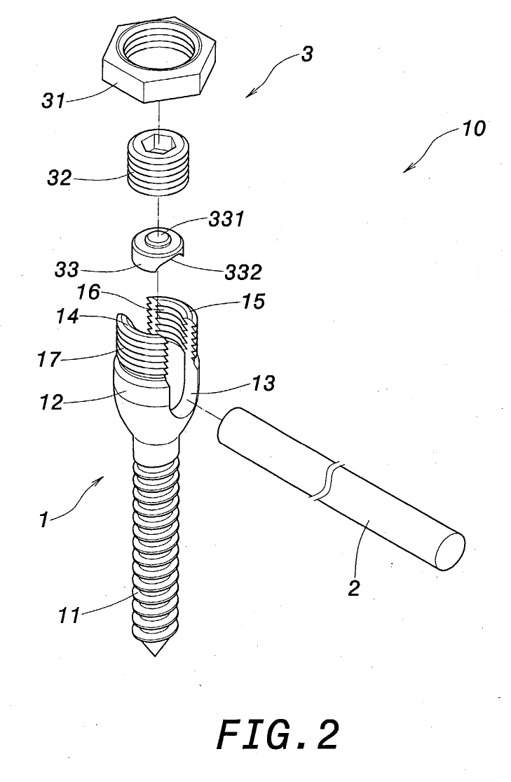

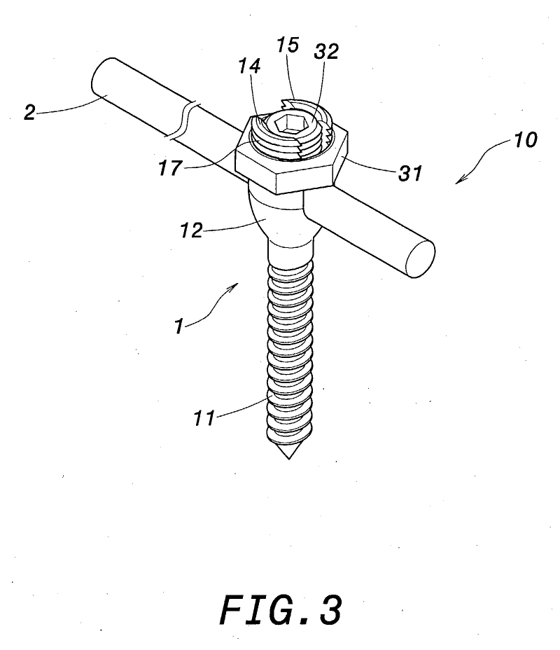

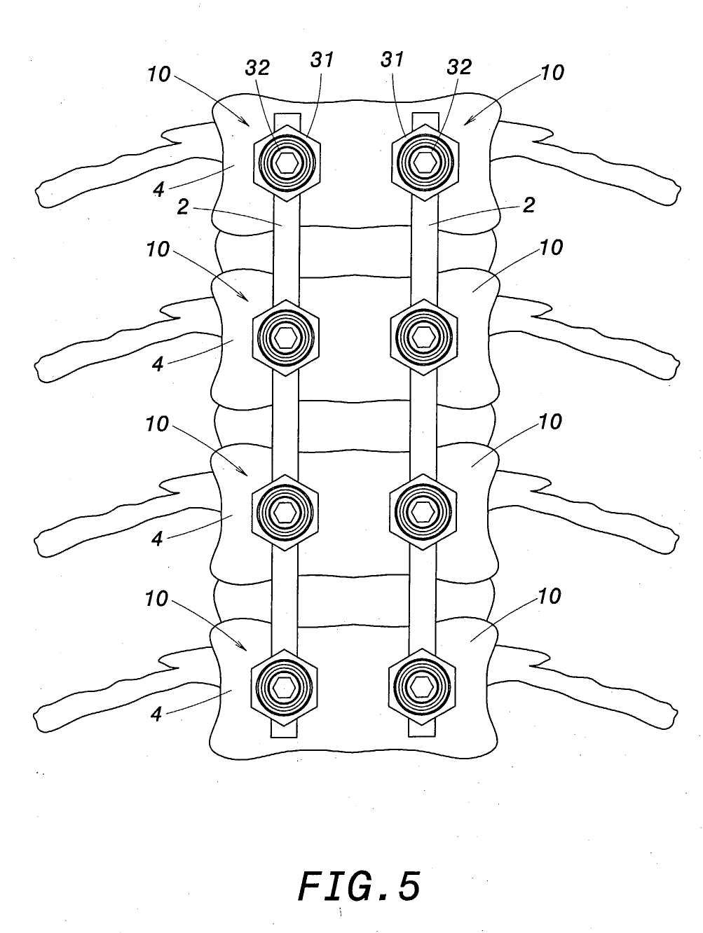

Please refer to FIG. 2 and FIG. 3, where FIG. 2 is an exploded diagram illustrating a spinal fixation device according to one preferred embodiment of this invention, and FIG. 3 is a perspective view of the spinal fixation assembly of FIG. 2. The present invention is directed to an improved structure of a multi-axis spinal fixation device. As shown in FIG. 2 and FIG. 3, the multi-axis spinal fixation device 10 comprises a supporting pole 1 comprising a threaded shaft 11 (as a bone screw) and a connecting seat 12. The threaded shaft 11 has buttress thread with a uniform outer diameter and conical type thread pattern. The inner diameter of the thread of the threaded shaft 11 increases from the tip of the threaded shaft 11 up to the connecting seat 12. As shown in FIG. 5, the threaded shaft 11 is used to implant into the spine 4. Due to the property of the conical thread, the bone of the spine surrounding the threaded shaft 11 of the supporting pole 1 generates a pre-stress effect, thereby increasing binding strength between the threaded shaft 11 and the bone. In addition, the buttress pattern of the threads helps to push the bone of the spine 4 between two threads. Increase of the cutting strength for damaging the structure of the bone is therefore needed. This amplifies the pre-stress effect and increases the axis-pulling strength, thereby stabilizing the spinal fixation device. In an osteoporosis case, the occlusion power gets larger since the pulling strength increases. The cylindrical connecting seat 12 has a U-shaped recess 13, which allows a brace member 2 or connecting rod to pass through or to rest thereon. The brace member 2 is typically an elongate cylindrical rod. The recess 13 is defined by the two uplifting flexible arms 14 and 15 formed at the upper portion of the connecting seat 12. Threads 16 and 17 are provided on the outer and inner surfaces of the two uplifting flexible arms 14 and 15.

A fastener 3 is fastened to the connecting seat 12. The fastener 3 comprises an outer nut 31 and an inner nut 32. The outer nut 31 has threads engaging with the threads formed on the outer surface of the two uplifting flexible arms 14 and 15, thereby restraining the two uplifting flexible arms 14 and 15. The inner nut 32 has threads on its periphery, which engage with the threads provided on the inner surface of the two uplifting flexible arms 14 and 15. The inner nut 32 is used to stabilize the brace rod 2 and restrain the brace rod 2 within the recess 13 of the supporting pole 1.

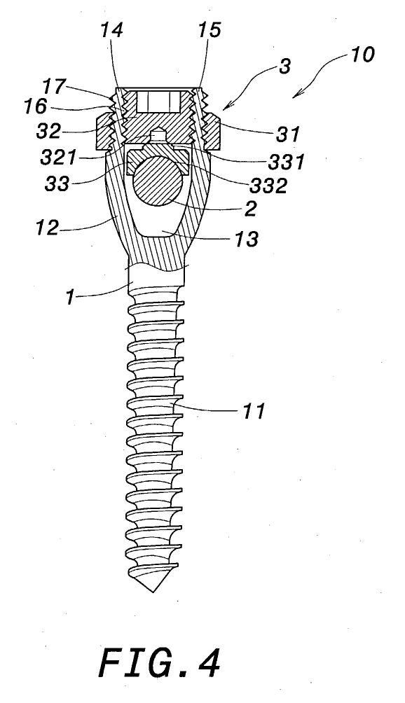

According to the present invention, the fastener 3 further comprises a stabilizing middle piece 33 located between the supporting pole 1 and inner nut 32. The stabilizing middle piece 33 has a protrusion 331 on its top surface facing the inner nut 32. The protrusion 331 inserts into the central aperture 321 of the inner nut 32, as indicated in FIG. 4. The stabilizing middle piece 33 has a curved surface 332 at its bottom side, which is engaged with the brace rod 2.

Please refer to FIG. 4. FIG. 4 is a cross-sectional diagram illustrating the spinal fixation device according to the present invention. When assembling the spinal fixation device 10, the brace member or brace rod 2 is first placed in the recess 13 of the supporting pole 1. Thereafter, the fastener 3 is fastened to the supporting pole 1. The stabilizing middle piece 33 is first placed in the recess 13 in the sense that the curved surface 332 of the stabilizing middle piece 33 leans against the surface of the brace rod 2. After installing the stabilizing middle piece 33, the inner nut 32 is screwed between the two flexible uplifting arms 14 and 15 to fasten the stabilizing middle piece 33. Finally, the outer nut 31 is screwed on the outer threads on the outer surface of the two flexible uplifting arms 14 and 15.

Please refer to FIG. 5. FIG. 5 is a plane view of the present invention when in use. When fixing two spines 4, the brace rod 2 is connected to at least two spinal fixation devices 10. The threaded shafts of the two spinal fixation devices 10 are implanted into the bone of the spines 4.

According to the present invention, it is advantageous to use the spinal fixation device since the curved surface 332 of the stabilizing middle piece 33 is in surface contact with the underlying brace rod 2, thereby tightly fastening the brace rod 2 on the supporting pole 1.

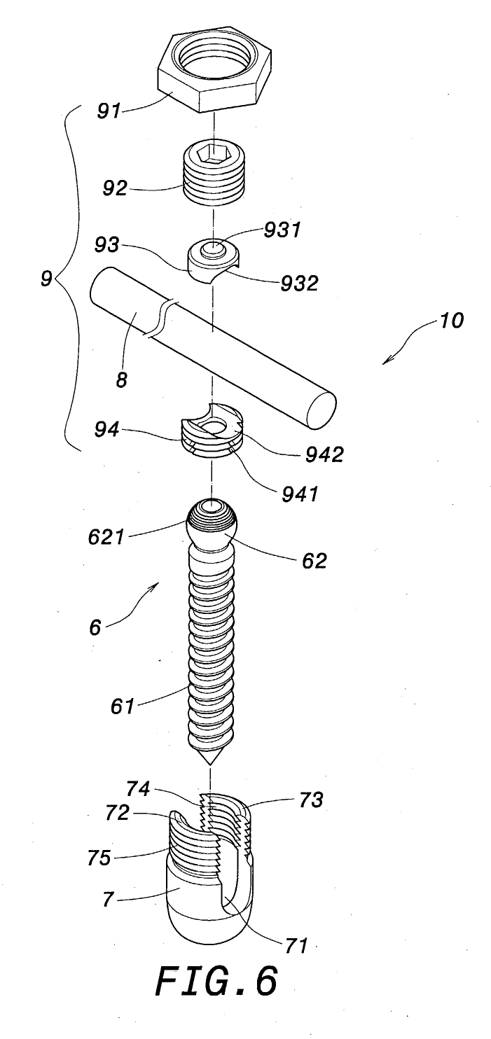

In the above-described embodiment, the supporting pole 1 and the connecting seat 12 are monolithic. That is, the supporting pole 1 cannot change its angle. Please refer to FIG. 6. FIG. 6 is another preferred embodiment according to the present invention. In this embodiment, the supporting pole 6 can change its implant angle. As shown in FIG. 6, the supporting pole 6 has a threaded shaft 61 for implanting the supporting pole 6 into the spine 4. The other end of the supporting pole 6 is a spherical connecting seat 62 having a roughed surface 621.

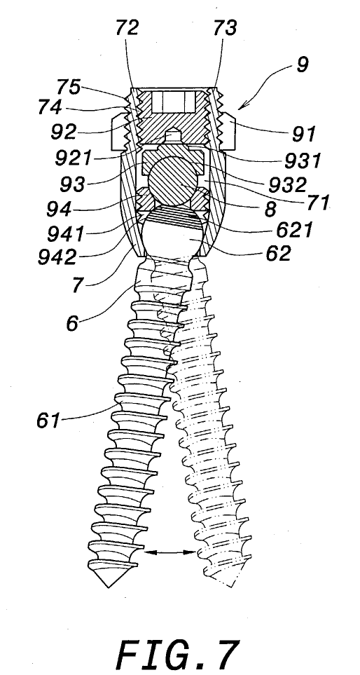

The spinal fixation device 10 according to the second preferred embodiment further comprises a hollow sleeve 7. The spherical connecting seat 62 of the supporting pole 6 is movably sleeved by the hollow sleeve 7, as shown in FIG. 7. At the upper end of the hollow sleeve 7, a U-shaped recess 71 is provided. The recess 71 allows a brace rod 8 to pass through or to rest thereon. Likewise, the recess 71 is defined by two uplifting flexible arms 72 and 73. Threads 74 and 75 are formed on inner and outer surfaces of the flexible arms 72 and 73. The diameter of an aperture at the tip of the hollow sleeve 7 is large enough such that the threaded shaft 61 of the supporting pole 6 can pass through. As specifically indicated in FIG. 7, the hallow sleeve 7 is tapered off so that the sleeve 7 can hold the spherical connecting seat 621.

A fastener 9 is provided to fasten the connecting seat 62 of the supporting pole 6 to the brace rod 8. The fastener 9, which is screwed to the hollow sleeve 7, comprises an outer nut 91 and an inner nut 92. The outer nut 91 engages with the threads formed on the outer surface of the two flexible arms 72 and 73 and thus restrains the two flexible arms 72 and 73. The inner nut 92 engages with the threads formed on the inner surface of the two flexible arms 72 and 73. The inner nut 92 is used to stabilize the underlying brace rod 8 and restrain the brace rod 8 within the recess 71 of the sleeve 7.

Please refer to FIG. 7. The fastener 9 further comprises an upper stabilizing piece 93 and a lower stabilizing piece 94. The upper stabilizing piece 93 is located between the brace rod 8 and the inner nut 92, and the lower stabilizing piece 94 is located between the brace rod 8 and the spherical connecting seat 62. As best seen in FIG. 7, the brace rod 8 is clamped by the upper stabilizing piece 93 and lower stabilizing piece 94. The upper stabilizing piece 93 has a protrusion 931 on its top surface facing the inner nut 92. The protrusion 931 inserts into the central aperture 921 of the inner nut 92, as indicated in FIG. 7. The upper stabilizing piece 93 has a curved surface 932 at its bottom side, which is engaged with the cylindrical brace rod 8 such that the upper stabilizing piece 93 is in surface contact with the brace rod 8. The lower stabilizing piece 94 has a plurality of trenches 941 disposed on its periphery corresponding to the connecting seat 62 of the supporting pole 6. The trenches 941 can increase the clamping force on the connecting seat 62 of the supporting-pole 6. The lower stabilizing piece 94 has a curved surface at its top end facing the brace rod 8 so that the lower stabilizing piece 94 is in surface contact with the brace rod 8.

Please still refer to FIG. 7. Since the connecting seat 62 and the supporting pole 6 is movably installed in the sleeve 7, the supporting pole 7 can thus move in multi-axis manner with the connecting seat 62 as axis center. When implanting the spinal fixation device into the spine, the implant angle can thus be adjusted. When adjusting the implant angle, the fastener 9 is first loosened such that a space is left between the lower stabilizing piece 94 and the supporting pole 6. An operator then adjusts the implant angle of the supporting pole 6, and then fastens the inner nut 92. Again, the lower stabilizing piece 94 will in contact with the connecting seat 62 of the supporting pole 6. Preferably, a roughed surface 621 is provided on the spherical connecting seat 62 to increase friction between the lower stabilizing piece 94 and the connecting seat 62.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A multi-axis spinal fixation device, comprising:
a supporting pole having a threaded shaft at one end and a connecting seat at the other, wherein the connecting seat comprises a U-shaped recess;
a brace rod installed across the U-shaped recess;
a fastener fastening the brace rod in the recess; and
a stabilizing piece disposed between the supporting pole and the fastener, the stabilizing piece having a curved surface at its one end facing the brace rod such that the stabilizing piece is in surface contact with the brace rod.

2. The multi-axis spinal fixation device as claimed in claim 1 wherein the recess is defined by two uplifting flexible arms having threads formed on their outer and inner surfaces.

3. The multi-axis spinal fixation device as claimed in claim 1 wherein the fastener comprises an outer nut and an inner nut, the outer nut engages with the threads formed outside the recess and the inner nut engages with the threads formed inside the recess.

4. The multi-axis spinal fixation device as claimed in claim 3 wherein the stabilizing piece has a protrusion on its top side facing the inner nut, the protrusion partially inserts into a central aperture of the inner nut.

5. The multi-axis spinal fixation device as claimed in claim 1 wherein the threaded shaft has buttress threads with a uniform outer diameter and conical type thread pattern, the inner diameter of the threads of the threaded shaft increases from the tip of the threaded shaft up to the connecting seat.

6. A multi-axis spinal fixation device, comprising:
a supporting pole having a threaded shaft at one end and a connecting seat at the other;
a hollow sleeve, having a recess at its upper end, used to movably sleeve the connecting seat and hold the connecting seat therein;
a brace rod laterally installed across the recess; and
a fastener screwed in the sleeve, thereby immobilizing the brace rod within the recess of the sleeve, wherein the fastener is in surface contact with the brace rod.

7. The multi-axis spinal fixation device as claimed in claim 6 wherein the connecting seat is spherical and has roughed surface.

8. The multi-axis spinal fixation device as claimed in claim 6 wherein the hallow sleeve is tapered off so that the sleeve can hold the connecting seat.

9. The multi-axis spinal fixation device as claimed in claim 6 wherein the recess is defined by two uplifting flexible arms having threads formed on their outer and inner surfaces.

10. The multi-axis spinal fixation device as claimed in claim 6 wherein the fastener further comprises an outer nut, an inner nut, an upper stabilizing piece, and a lower stabilizing piece, the upper stabilizing piece is located between the brace rod and the inner nut, and the lower stabilizing piece is located between the brace rod and the connecting seat, the brace rod is clamped by the upper stabilizing piece and lower stabilizing piece, the upper and lower stabilizing pieces have curved surface, which are engaged with the brace rod such that the upper and lower stabilizing pieces are in surface contact with the brace rod.

11. The multi-axis spinal fixation device as claimed in claim 10 wherein the upper stabilizing piece has a protrusion on its top surface facing the inner nut, the protrusion inserts into the central aperture of the inner nut.

12. The multi-axis spinal fixation device as claimed in claim 10 wherein the lower stabilizing piece has a plurality of trenches disposed on its periphery corresponding to the connecting seat of the supporting pole.

13. The multi-axis spinal fixation device as claimed in claim 6 wherein the threaded shaft has buttress threads with a uniform outer diameter and conical type thread pattern, the inner diameter of the threads of the threaded shaft increases from the tip of the threaded shaft up to the connecting seat.
